(19) United States
(12) Patent Application Publication
(10) Pub. No.: US 2013/0131666 A1
(43) Pub. Date: May 23, 2013

(54) ELECTROSURGICAL INSTRUMENT

(71) Applicant: Gyrus Medical Limited, St Mellons (GB)

(72) Inventors: Anthony K. ATWELL, Newport (GB); Marno NAGTEGAAL, Cardiff (GB); Chun Keat OOI, Cardiff (GB)

(73) Assignee: GYRUS MEDICAL LIMITED, St Mellons (GB)

(21) Appl. No.: 13/675,478

(22) Filed: Nov. 13, 2012

(30) Foreign Application Priority Data
Nov. 18, 2011 (GB) 1119897.5

(51) Int. Cl. A61B 18/14; A61B 17/00

(52) U.S. Cl. CPC A61B 18/1445; A61B 17/00234
USPC 606/41; 606/206

(57) ABSTRACT

An endoscopic surgical instrument includes a stationary component, an elongate shaft extending from the stationary component, and an end effector located at the distal end of the shaft. The stationary component includes at least one contact portion adapted to be engaged by the fingers of a user, the instrument also including a movable component including a contact portion adapted to be engaged by the thumb of a user. Longitudinal movement between the contact portions of the movable component and the stationary component cause the movable component to move longitudinally with respect to the stationary component between a first proximal position, a second distal position, and a third position, the third position being distal of the second position by a relatively small distance. The movement of the movable component between the first and second positions causes a corresponding movement of the end effector between corresponding first and second positions.

This invention relates to an electrosurgical instrument for the treatment of tissue. Such instruments are commonly used for the cutting/vaporisation and/or desiccation/coagulation of tissue in surgical intervention, most commonly in “keyhole” or minimally invasive surgery. The terms “cutting” and “vaporization” relate to the removal of tissue, whether by resection or by the volumetric removal of tissue. Similarly, the terms “desiccation” and “coagulation” relate to the creation of lesions in tissue, the necrosis of tissue, and to the prevention of bleeding.

Endoscopic instruments are often used in gastroenterology or cardiac surgery, and such instruments are normally introduced through an endoscope working channel, with the endoscope in turn introduced through a lumen within the patient's body. These instruments are therefore of a relatively small size, often no more than 5 mm in diameter. They are deployed at the end of a relatively long flexible shaft, such that they can be maneuvered within a lumen as described above.

Where such instruments include the deployment of one component relative to another, such deployment is often carried out by sliding one handle component relative to another. The components are often supplied with loops or moulded surfaces adapted to be contacted by the fingers and thumb of the user of the instrument. One instrument of this type is described in U.S. Pat. No. 5,290,286 in which a component is movable within a housing, the movable component being provided with a thumb ring and the housing with finger loops. The present invention attempts to provide an improvement to endoscopic instruments of this type.

Accordingly, an endoscopic surgical instrument is provided including a stationary component, an elongate shaft extending from the stationary component, and an end effector located at the distal end of the shaft, the stationary component including at least one contact portion adapted to be engaged by the fingers of a user, the instrument also including a movable component including a contact portion adapted to be engaged by the thumb of a user, longitudinal movement between the contact portions of the movable component and the stationary component causing the movable component to move longitudinally with respect to the stationary component between a first proximal position, a second distal position, and a third position, the third position being distal of the second position by a relatively small distance, the movement of the movable component between the first and second positions causing a corresponding movement of the end effector between first and second end effector positions, the instrument further including a biasing mechanism to urge the movable component towards the first position, and a latch mechanism such that when the movable component is initially moved from the first position to the third position, the latch mechanism is engaged such that when the movable component returns to the second position under the action of the biasing mechanism, the movable component is held in to the second position by the latch mechanism.

The term “stationary component” does not mean that the component is immobile, as the component can be moved in order to manoeuvre the shaft and end effector into position within the body of a patient. However, when the surgical instrument is in position and the user wishes to activate the end effector, the stationary component remains in position and the movable component moves with respect thereto. It is therefore the movement of the movable component that causes the end effector to be activated.

The stationary component and the movable component form a handle mechanism which can be operated by the fingers and thumb of one hand. In this way, a movable component (such as a piston) can be manipulated with respect to the stationary component by a direct sliding action of the contact portions one with respect to another. This is distinguished from other types of handle mechanism in which one arm pivots with respect to another, in a scissors-type arrangement. In the direct sliding mechanism, the movement of the movable component can be accomplished by movement between the fingers and thumb of one hand. However, once the movable component has been moved in to its (deployed) second position, the latch mechanism ensures that it remains in that position until the latch mechanism is released. This is in contrast to prior art devices, which require the surgeon (or an assistant) to hold the handle in position to ensure that the instrument remains deployed.

As previously mentioned, the instrument contains a biasing mechanism to urge the movable component towards the first position. Where the first position is the undeployed or “at rest” position and the second position is the deployed position, it is often convenient to bias the instrument into the un-deployed position, requiring a force to be overcome in order to deploy the instrument. However, the latching mechanism ensures that once the instrument has been deployed, it can be locked in its deployed position allowing the user to relax rather than need to continue to overcome the biasing force to maintain the instrument in its deployed position.

The latch mechanism is conveniently such that when the movable component is subsequently moved from the second position to the third position, the latch mechanism is disengaged such that the movable component can return to the first position under the action of the biasing mechanism. In this way, when the user wishes to deploy the instrument or activate the end effector, the user moves the handle such that the movable component moves from the first position to the second position. The user continues to move the movable component until it reaches the third position (which is typically only a small amount further than the second position). The user then releases the handle, and the movable component moves back to the second position under the influence of the biasing mechanism, where it is held by the latching mechanism.

When the user wishes to retract the instrument or return the end effector to its original position, the user once again moves the handle such that the movable component once again moves to the third position. Now, when the handle is released, the latching mechanism is released and the movable component moves back to the first position. The latching mechanism is therefore a “click-on-click-off” mechanism, somewhat similar in operation to that of a retractable ballpoint pen. The stationary component conveniently contains the biasing mechanism and the latch mechanism.

The latch mechanism is preferably constituted by a cam follower on either the stationary component or the movable component, engaging with a cam track on the other of the stationary component or the movable component. Typically, the cam track is present on the movable component, and the cam follower on the stationary component. The cam arrangement locks and unlocks the movable component with respect to the stationary component automatically as the movable component moves between its first and second positions.

According to a convenient arrangement, the cam follower is located on a cantilever arm one end of which is fixed to the component on which the cam follower is provided. The cam track is preferably provided with a first pathway which the cam follower follows when the movable component is moved from the first position to the second position, and a second pathway which the cam follower follows when the movable component is moved from the second position to the first position. In this way, the cam follower follows one pathway when the instrument is deployed, and a different pathway when the latch is being released and the instrument retracted. The cam track is conveniently provided with a V-shaped section to ensure that the cam follower follows the second pathway rather than the first pathway when the movable component is moved from the second position to the first position. The cam follower enters the V-shaped section in one direction as the instrument is being deployed, and leaves by a different direction as the instrument is being retracted.

According to a convenient construction, the stationary component comprises a housing, and the movable component moves within the housing. Typically, the stationary component includes a cylindrical bore and the movable component is a piston located within the cylindrical bore. The instrument is therefore somewhat similar to a syringe, with a piston sliding within the cylindrical bore in order to activate the end effector.

The instrument, preferably the movable component, may include a lost motion mechanism arranged such that movement of the movable component between the second and third positions does not cause a corresponding movement of the end effector. The main purpose of the lost motion mechanism is to limit the force applied to the end effector. This lost motion mechanism typically includes a spring located within the movable component such that movement of the movable component between the second and third positions causes a compression or relaxation of the spring rather than a movement of the end effector. The end effector is controlled by limit stops at the distal end of the instrument. The lost motion mechanism prevents the limit stop mechanism from being overloaded. This lost motion mechanism ensures that the movable component can be moved from the second position to the third position in order to release the latching mechanism without causing any unwanted movement of the end effector.

Similarly, the instrument, preferably the movable component, also conveniently includes a second lost motion mechanism arranged such that movement of the movable component between the first position and an intermediate position part way between the first and second positions does not cause a corresponding movement of the end effector. Once again, this lost motion mechanism typically includes a spring located within the movable component such that movement of the movable component between the first position and the intermediate position causes a compression or relaxation of the spring rather than a movement of the end effector. This ensures that the end effector is only activated by a positive and sustained movement of the movable component from its first position towards its second position, and not by any minor movement such as that possibly generated by movement of the endoscope, or by other bumps and jolts. Typically, the lost motion mechanism comprises first and second springs located within a single bore within the movable component, thereby providing the desired effect for both the first and second lost motion mechanisms.

More particularly, in the preferred instrument, the movable component comprises first and second parts which can slide relatively to each other in the longitudinal direction, the first part having a contact portion for the user's thumb and the second part being coupled to the end effector, wherein the movable component has a longitudinal bore and comprises first and second springs located within the bore to bias the second part of the movable component longitudinally relative to the first part of the movable component in respective directions, the arrangement acting as a lost motion mechanism which operates such that movement of the movable component between its second and third positions is accommodated by the first spring rather than causing a movement of the end effector, and such that movement of the movable component between its first position and an intermediate position part-way between its first and second positions is accommodated by the second spring rather than causing a movement of the end effector.

According to one convenient arrangement, the end effector comprises a pair of jaws, the jaws being open when the movable component is in the first position and the jaws being closed when the movable component is in the second position. This is typical for when the instrument is a forceps device, with jaws capable of grasping and manipulating tissue. Alternatively, the end effector comprises an electrode movable between a deployed position and a retracted position, the electrode being in its retracted position when the movable component is in the first position and electrode being in its deployed position when the movable component is in the second position. This is typical when the instrument is an electrosurgical instrument, with an electrode capable of being deployed or retracted and capable of either the vaporisation or coagulation of tissue.

The invention further resides in a handle for an endoscopic surgical instrument, the handle including a stationary component and a movable component, the stationary component including at least one contact portion adapted to be engaged by the fingers of a user, the movable component including a contact portion adapted to be engaged by the thumb of a user, longitudinal movement between the contact portions of the movable component and the stationary component causing the movable component to move longitudinally with respect to the stationary component between a first proximal position, a second distal position, and a third position, the third position being distal of the second position by a relatively small distance, the instrument further including a biasing mechanism to urge the movable component towards the first position, and a latch mechanism such that when the movable component is initially moved from the first position to the third position, the latch mechanism is engaged such that when the movable component returns to the second position under the action of the biasing mechanism, the movable component is held in the second position by the latch mechanism.

The latch mechanism is conveniently such that when the movable component is subsequently moved from the second position to the third position, the latch mechanism is disengaged such that the movable component can return to the first position under the action of the biasing mechanism.

The invention will now be further described, by way of example only, with reference to the accompanying drawings, in which:

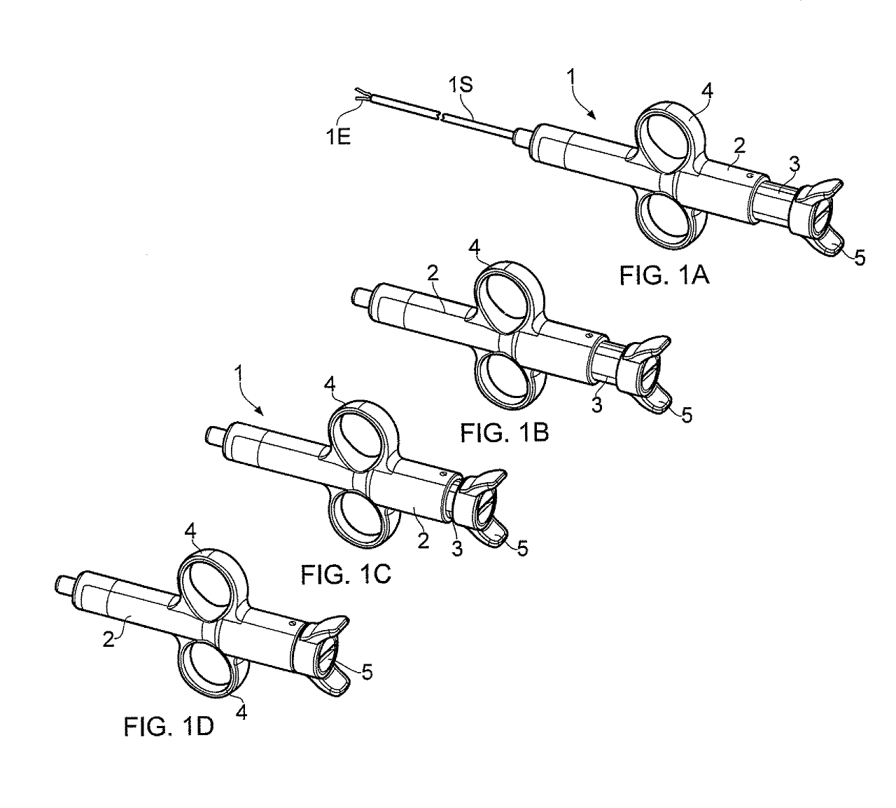

FIGS. 1A to 1D are perspective views of the handle of a surgical instrument in accordance with the present invention, shown in different positions,

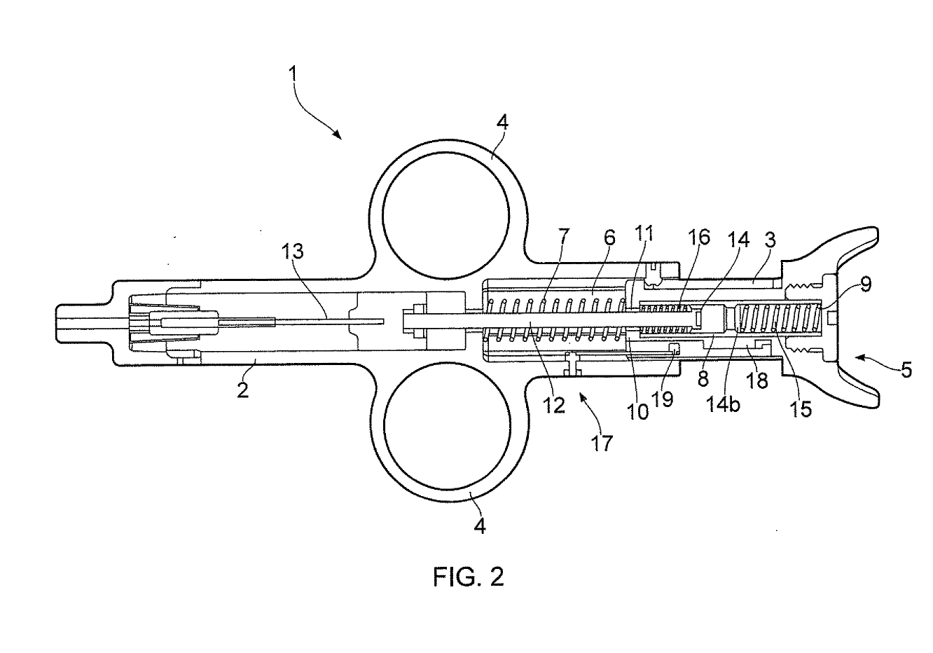

FIG. 2 is a sectional side view of the electrosurgical instrument of FIGS. 1A to 1D, shown in the position of FIG. 1A,

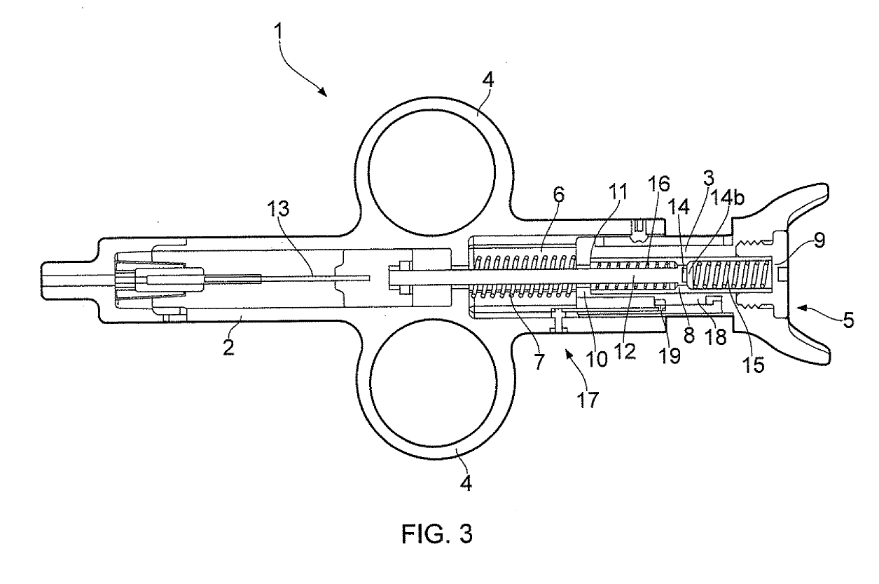

FIG. 3 is a sectional side view of the electrosurgical instrument of FIGS. 1A to 1D, shown in the position of FIG. 1B,

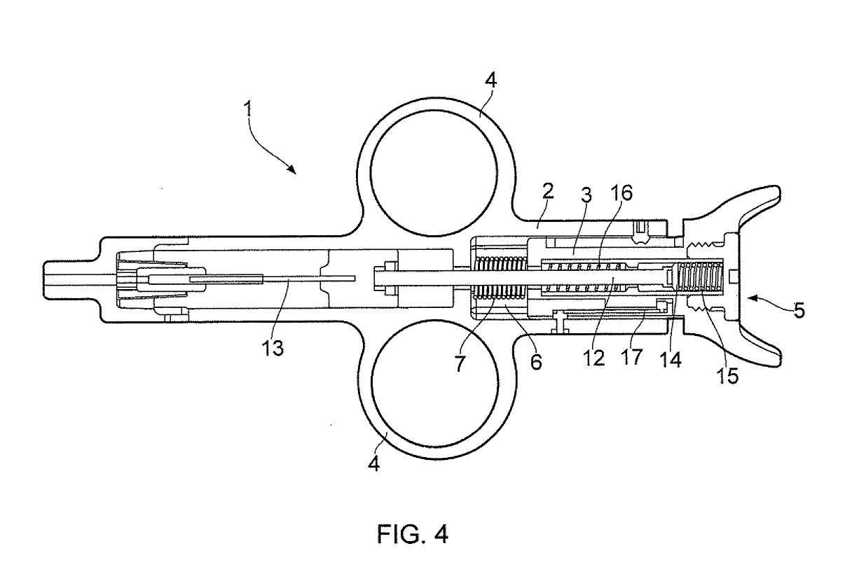

FIG. 4 is a sectional side view of the electrosurgical instrument of FIGS. 1A to 1D, shown in the position of FIG. 1C,

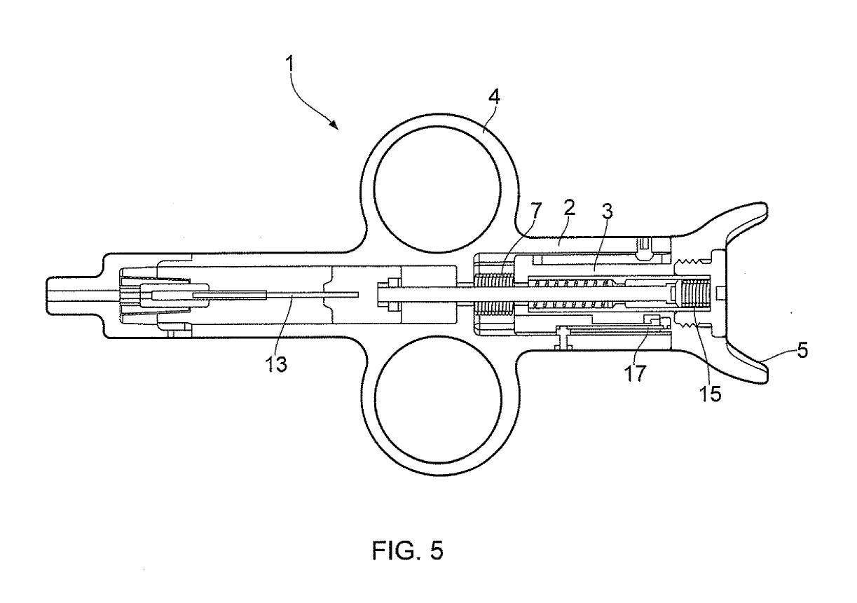

FIG. 5 is a sectional side view of the electrosurgical instrument of FIGS. 1A to 1D, shown in the position of FIG. 1D,

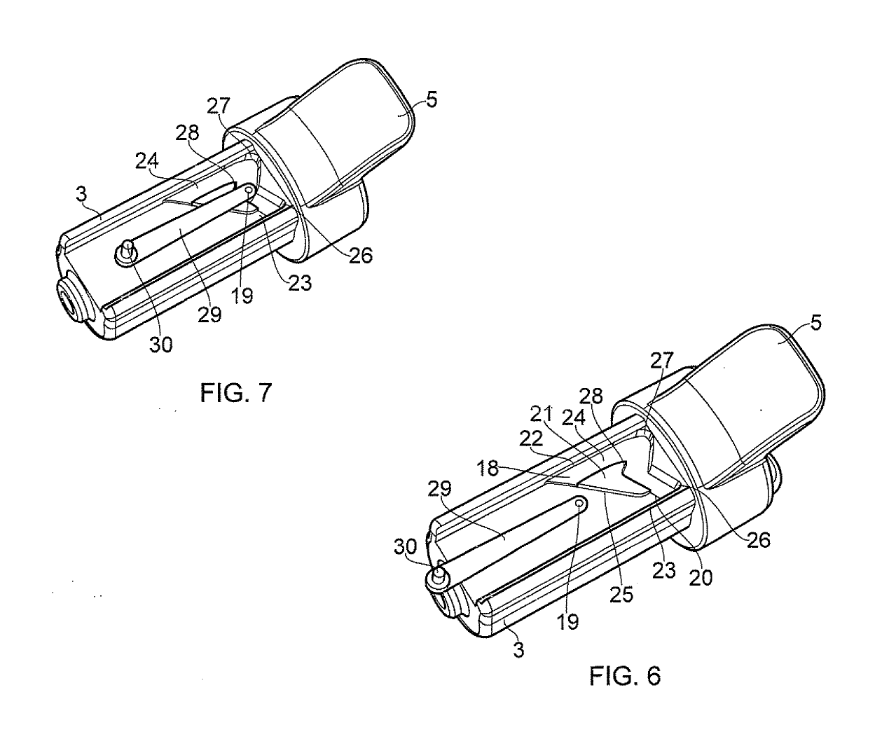

FIG. 6 is an exploded view of one of the components of the instrument of FIGS. 1A to 1D, shown in the position of FIG. 1A, and

FIG. 7 is an exploded view of the component of FIG. 5, shown in the position of FIG. 1C,

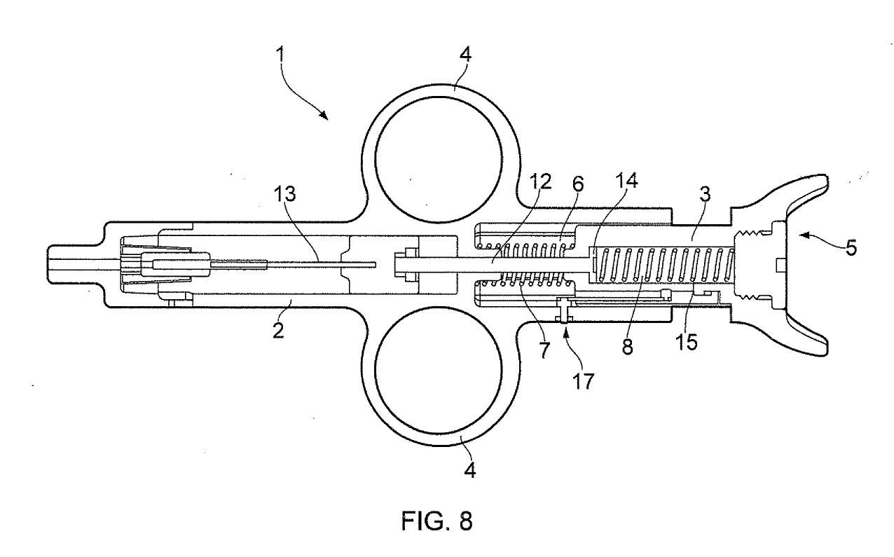

FIG. 8 is a sectional side view of an alternative embodiment of electrosurgical instrument, shown in a position equivalent to that of FIG. 1B,

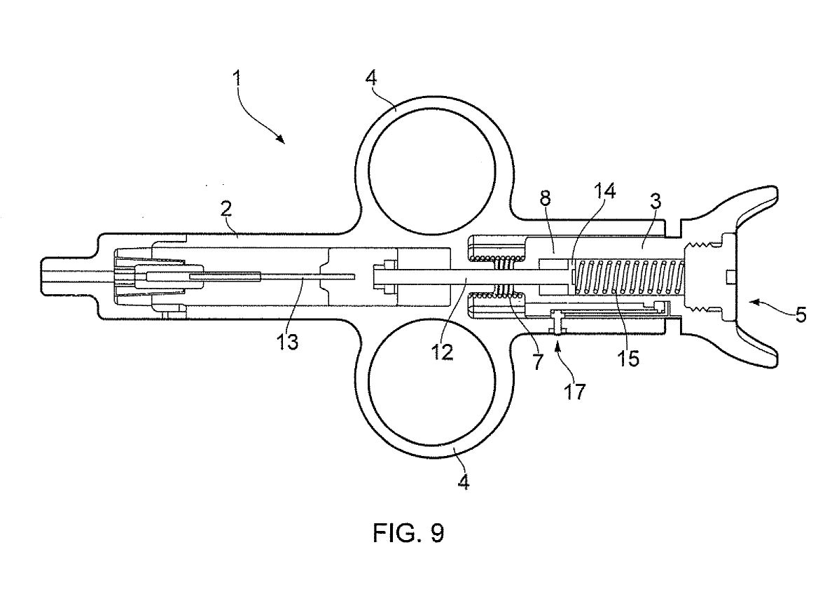

FIG. 9 is a sectional side view of the electrosurgical instrument of FIG. 8, shown in a position equivalent to that of FIG. 1C,

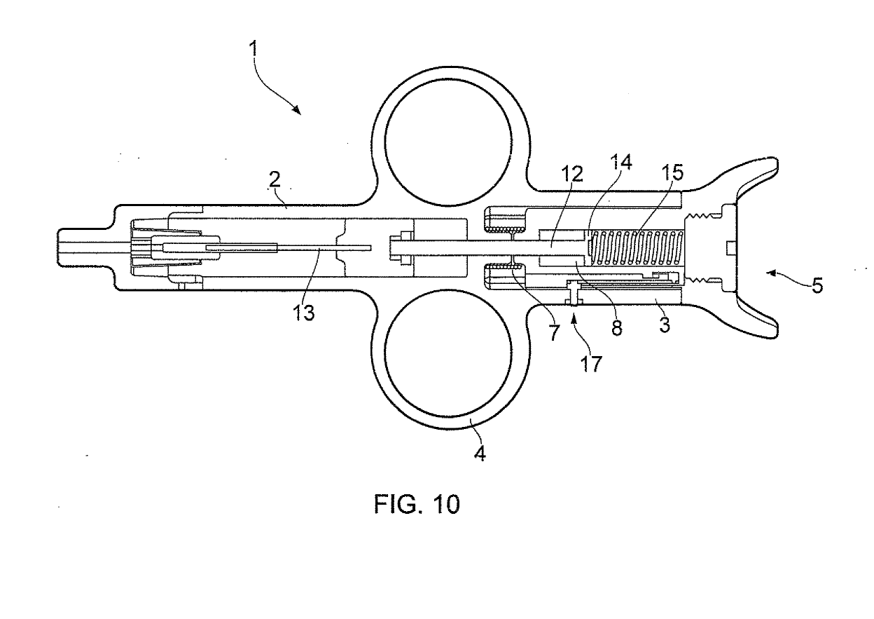

FIG. 10 is a sectional side view of the electrosurgical instrument of FIG. 8, shown in a position equivalent to that of FIG. 1D,

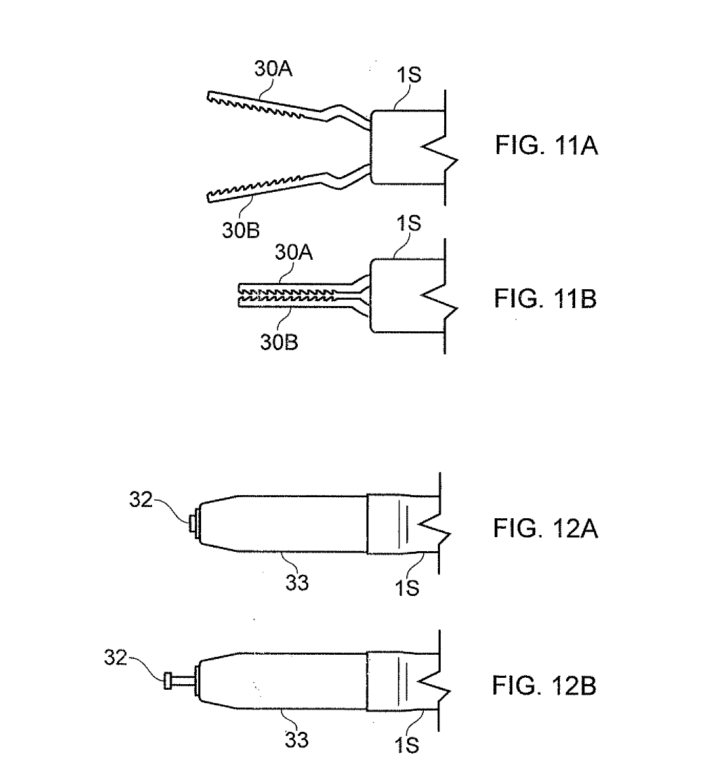

FIGS. 11A and 11B are side views of a distal end portion of an instrument shaft with an end effector in the form of a pair of jaws, and

FIGS. 12A and 12B are side views of a distal end portion of an alternative instrument shaft with an end effector in the form of an extendable electrode.

Referring to FIG. 1A, an endoscopic surgical instrument in accordance with the invention has a handle shown generally at 1, an instrument shaft 1S and an end effector 1E, in this example a jaw mechanism. The shaft 1S is shown in a diagrammatic broken view in FIG. 1A as normally it is longer than can be shown to scale in the drawing. The handle 1 includes a stationary component in the form of a cylindrical housing 2 and a movable component in the form of a piston 3, the piston being slideable within the housing 2. A pair of finger loops 4 is present on the housing 2, and a cradle 5 is present at the proximal end of the piston 3, the cradle being adapted to receive the thumb of a user of the instrument. By holding the finger loops 4 and pushing the thumb cradle 5, the user can move the piston 3 into the housing 2. FIG. 1A shows the handle in a first or “retracted” position, in which the piston 3 extends out of the housing 2. In FIGS. 1B to 1D, which show the handle in different conditions, the shaft and end effector are omitted for simplicity. FIG. 1B shows the handle in a partly deployed position, in which the piston 3 is being inserted part way into the housing. FIG. 1C shows the handle in a “deployed” position, in which the piston 3 has been inserted into the housing 2. FIG. 1D shows the handle in a final position, which is a temporary “over-extended” position, used to release a latching mechanism which will be described shortly.

FIG. 2 shows the handle in the retracted position of FIG. 1A. The piston 3 is received in a cylindrical bore 6 within the housing 2, and biased into the retracted position by a first spring 7 located in the bore. The piston 3 itself has a cylindrical chamber 8, with a proximal end wall 9, a distal end wall 10, and an aperture 11 in the distal end wall 10. A push rod 12 is present within the chamber 8, extending distally through the aperture 11, where it is connected to a smaller diameter push rod 13 extending from the distal end of the handle 1 along the shaft (not shown in FIG. 2) at the end of which is an end effector (also not shown) forming the tissue-treatment part of the surgical instrument. The push rod 12 terminates at its proximal end in an end plate 14 located part-way within the chamber 8. A second spring 15 is present between the washer 14b and the proximal end wall 9 of the chamber 8, while a third spring 16 is present between the end plate 14 and the distal end wall 10 of the chamber 8. As the force exerted by spring 7 exceeds the force exerted by spring 16, the washer 14b is spaced from the end plate 14 as shown in FIG. 2.

A latch mechanism 17 is provided within the housing 2, and comprises a cam track 18 present on the piston 3 and a cam follower 19 associated with the housing 2. The operation of the latch mechanism will be described in more detail subsequently.

To operate the surgical instrument, a user grips the handle 1 with fingers placed within the finger loops 4 and depresses the piston 3 using the thumb cradle 5. The piston 3 moves distally within the bore 6, but at first this will merely close the gap between washer 14b and end plate 14, and will not cause any corresponding movement of the push rod 12 (and hence the end effector of the instrument) will not be moved. However, once the gap between the washer 14b and the end plate 14 has been closed, as shown in FIG. 3, further movement of the piston 3 will cause the push rod 12 to slide within the housing 2 to the position shown in FIG. 4. In FIG. 4, the piston 3 is in the deployed position of FIG. 1C, with the piston almost completely contained within the housing 2. The piston has moved along the bore 6 against the action of the spring 7, which is still attempting to move the piston back to the position shown in FIG. 2. At this point, the latch mechanism 17 has not yet operated, such that if the pressure on the thumb cradle 5 is relaxed, the piston will return to the position shown in FIG. 2, under the action of the spring 7.

To operate the latch mechanism 17, the user continues to depress the piston 3 until it reaches the “over-extended” position shown in FIG. 5. This further movement of the piston is fully taken up by the spring 15, and so will not move the push rod 12 (and hence the end effector) any further. However, this additional movement of the piston is sufficient to operate the latch mechanism 17, as will be described in more detail subsequently. With the latch mechanism 17 engaged, the user is able to remove the pressure on the thumb cradle 5, and the piston will only move back as far as the deployed position shown in FIG. 1C and FIG. 4, with the latch mechanism 17 preventing any further proximal movement of the piston. In this position, the end effector is in its deployed position, such that the surgical intervention can be carried out.

When the user wishes to return the end effector to its “retracted” position, the user once again depresses the piston 3 to move it to the “over-extended” position of FIG. 1D and FIG. 5. This movement disengages the latch mechanism 17, such that when the user subsequently removes the pressure on the thumb cradle 5, the piston returns under the action of spring 7 all the way back to the position shown in FIG. 1A and FIG. 2. In this way, the user is able to deploy and retract the piston 3, and hence the end effector, with confidence, using a “click-on, click-off” method of operation. With the latch mechanism 17 holding the piston in its deployed position, there is no need for the user or an assistant to hold the handle 1 to ensure that the piston does not retract before it is required to do so.

FIGS. 6 and 7 show the operation of the latch mechanism 17. FIG. 6 shows the cam track 18 present on the piston 3. The cam track 18 is provided by means of a channel 20 formed between a raised moulded component 21 and an outer wall 22. The cam track includes first and second pathways 23 24, on opposite sides of the moulded component, together with a sloping proximal section 25, and a V-shaped end section including first and second extremities 26, 27 and a notch 28 therebetween. The cam follower 19 is present at the end of a cantilever aim 29, the proximal end of the cantilever arm being a pivot point 30 fixed to the housing 2.

FIG. 6 shows the position of the latch mechanism when the piston is in the retracted position. The cam follower 19 is adjacent the sloping section 25, which urges the cam follower towards the first pathway 23. As the piston is moved into the housing, the cam follower 19 moves along the first pathway 23 until it reaches the first extremity 26, which is reached when the piston 3 reaches its “over-extended” position as shown in FIG. 1D and FIG. 5. When the pressure on the thumb cradle is relaxed, the cam follower 19 moves into the notch 28, locking the piston 3 in position within the housing 2 in the “deployed” position shown in FIG. 1C and FIG. 4. This is the position shown in FIG. 7.

To disengage the latch mechanism 17, the user depresses the piston 3 once more, to move the piston once again into the “over-extended” position. This time the cam follower 19 moves out of the notch 28 and into the second extremity 27. When the pressure on the thumb cradle 5 is released, the cam follower moves back along second pathway 24 until it returns to the position shown in FIG. 6, ready for the process to be repeated if required. As can be seen, the latch mechanism 17 provides a “click-on, click-off” method of operation, in which one push of the piston 3 engages the latch mechanism and another push disengages it. The surgical instrument can therefore be locked into its deployed position, merely by depressing and releasing the piston 3, and with no requirement for other buttons or levers to be activated. Similarly, the surgical instrument can be unlocked and returned to its retracted position, again merely by depressing and releasing the piston a second time, and once again with no requirement for any additional locking buttons or levers to be activated.

FIG. 8 shows an alternative embodiment of surgical instrument which uses only springs 7 and 15, and dispenses with spring 16. This instrument does not have the fully retracted position of FIG. 1A or FIG. 2, and the retracted position is as shown in FIG. 8. In this arrangement, the push rod 12 (and hence the end effector) will start to move as soon as the piston 3 is depressed. The latch mechanism 17 and other components are as previously described, and so the instrument will be latched into the deployed position as shown in FIG. 9, exactly as before. It will be noted that the movement of the piston between the positions shown in FIGS. 9 and 10 causes a compression of the spring 15 rather than further movement of the push rod 12. FIG. 10 shows the “over-extended” position used to release the latch mechanism 17, such that the spring 7 can urge the piston 3 back to the position as shown in FIG. 8.

Other variations can be envisaged without departing from the scope of the present invention. For example, a variety of latching mechanisms can be employed, either with different cam and cam follower arrangements, or using known latching mechanisms not employing a cam arrangement.

The end effector can be any of a range of known surgical treatment devices, as long as some form of movement is required. These include jaw mechanisms, articulating mechanisms, and extendable electrodes or other components. Referring to FIGS. 11A and 11B, a jaw mechanism typically comprises a pair of jaws 30A, 30B which, in a first end effector position, are open as shown in FIG. 11A, that is when the piston 3 of the handle 1 is in its retracted position as shown, for instance, in FIG. 1A. When the piston 3 is pushed into the housing of the handle 1 so that it reaches its second, deployed position, the pushrod 12 (FIG. 2), which extends into the shaft 1S, closes the jaws 30A, 30B, as shown in FIG. 11B.

In an alternative end effector, comprising a retractable and extenable electrode 32 as shown in FIGS. 12A and 12B, depression of the piston 3 moves the pushrod 12 in the shaft 1S as described above to move the electrode 32 from its retracted position within a nose portion 33 of the shaft 1S, as shown in FIG. 12A, to its extended position, as shown in FIG. 12B.

The handle of the present invention is suitable for any of a range of such devices. Similarly, other finger and thumb grip constructions can be envisaged, without departing from the scope of the present invention.

1. An endoscopic surgical instrument including a stationary component, an elongate shaft extending from the stationary component, and an end effector located at the distal end of the shaft, the stationary component including at least one contact portion adapted to be engaged by the fingers of a user, the instrument also including a movable component including a contact portion adapted to be engaged by the thumb of a user, longitudinal movement between the contact portions of the movable component and the stationary component causing the movable component to move longitudinally with respect to the stationary component between a first proximal position, a second distal position, and a third position, the third position being distal of the second position by a relatively small distance, the movement of the movable component between the first and second positions causing a corresponding movement of the end effector between first and second end effector positions, the instrument further including a biasing mechanism to urge the movable component towards its first position, and a latch mechanism such that when the movable component is initially moved from its first position to its third position, the latch mechanism is engaged such that when the movable component returns to its second position under the action of the biasing mechanism, the movable component is held in its second position by the latch mechanism.

2. An endoscopic surgical instrument according to claim 1, wherein the latch mechanism is such that when the movable component is subsequently moved from its second position to its third position, the latch mechanism is disengaged such that the movable component can return to its first position under the action of the biasing mechanism.

3. An endoscopic surgical instrument according to claim 1, wherein the stationary component contains the biasing mechanism.

4. An endoscopic surgical instrument according to claim 1, wherein the stationary component contains the latch mechanism.

5. An endoscopic surgical instrument according to claim 1, wherein the latch mechanism is constituted by a cam follower on one of the stationary or movable components, engaging with a cam track on the other of the stationary or movable components.

6. An endoscopic surgical instrument according to claim 5, wherein the cam track is present on the movable component, and the cam follower on the stationary component.

7. An endoscopic surgical instrument according to claim 5, wherein the cam follower is located on a cantilever arm one end of which is fixed to the component on which the cam follower is provided.

8. An endoscopic surgical instrument according to claim 5, wherein the cam track is provided with a first pathway which the cam follower follows when the movable component is moved from the first position to the second position, and a second pathway which the cam follower follows when the movable component is moved from the second position to the first position.

9. An endoscopic surgical instrument according to claim 8, wherein the cam track is provided with a v-shaped section to ensure that the cam follower follows the second pathway rather than the first pathway when the movable component is moved from the second position to the first position.

10. An endoscopic surgical instrument according to claim 1, wherein the stationary component comprises a housing, and the movable component moves within the housing.

11. An endoscopic surgical instrument according to claim 10, wherein the housing includes a cylindrical bore and the movable component is a piston located within the cylindrical bore.

12. An endoscopic surgical instrument according to claim 1, including a lost motion mechanism arranged such that movement of the movable component between the second and third positions does not cause a corresponding movement of the end effector.

13. An endoscopic surgical instrument according to claim 12, wherein the lost motion mechanism comprises a spring located such that movement of the movable component between the second and third positions is accommodated by the spring rather than causing a movement of the end effector.

14. An endoscopic surgical instrument according to claim 1, including a lost motion mechanism arranged such that movement of the movable component between the first position and an intermediate position part way between the first and second positions does not cause a corresponding movement of the end effector.

15. An endoscopic surgical instrument according to claim 14, wherein the lost motion mechanism comprises a spring located such that movement of the movable component between the first position and the intermediate position is accommodated by the spring rather than causing a movement of the end effector.

16. An endoscopic surgical instrument according to claim 1, wherein the movable component comprises first and second parts which can slide relatively to each other in the longitudinal direction, the first part having a contact portion for the user's thumb and the second part being coupled to the end effector, wherein the movable component has a longitudinal bore and comprises first and second springs located within the bore to bias the second part of the movable component longitudinally relative to the first part of the movable component in respective directions, the arrangement acting as a lost motion mechanism which operates such that movement of the movable component between its second and third positions is accommodated by the first spring rather than causing a movement of the end effector, and such that movement of the movable component between its first position and an intermediate position part-way between its first and second positions is accommodated by the second spring rather than causing a movement of the end effector.

17. An endoscopic surgical instrument according to claim 1, wherein the end effector comprises a pair of jaws, the jaws being open when the movable component is in the first end effector position and the jaws being closed when the movable component is in the second end effector position.

18-21. (canceled)

22. An endoscopic surgical instrument according to claim 1, wherein the end effector comprises an electrode movable between a deployed position and a retracted position, the electrode being in its retracted position when the movable component is in its first position and electrode being in its deployed position when the movable component is in its second position.

23. A handle for an endoscopic surgical instrument, the handle including a stationary component and a movable component, the stationary component including at least one contact portion adapted to be engaged by the fingers of a user, the movable component including a contact portion adapted to be engaged by the thumb of a user, longitudinal movement between the contact portions of the movable component and the stationary component causing the movable component to move longitudinally with respect to the stationary component between a first proximal position, a second distal position, and a third position, the third position being distal of the second position by a relatively small distance, the instrument further including a biasing mechanism to urge the movable component towards its first position, and a latch mechanism such that when the movable component is initially moved from its first position to its third position, the latch mechanism is engaged such that when the movable component returns to the second position under the action of the biasing mechanism, the movable component is held in the second position by the latch mechanism.

24. A handle according to claim 23, wherein the latch mechanism is such that when the movable component is subsequently moved from the second position to the third position, the latch mechanism is disengaged such that the movable component can return to the first position under the action of the biasing mechanism.
